(19) United States
(12) Patent Application Publication
(10) Pub. No.: US 2023/0377153 A1
(43) Pub. Date: Nov. 23, 2023

(54) SYSTEMS AND METHODS FOR TISSUE EVALUATION AND CLASSIFICATION

(71) Applicant: OneProjects Design and Innovation Ltd., Dublin (IE)

(72) Inventor: Christoph Hennersperger, München (DE)

(21) Appl. No.: 18/198,538

(22) Filed: May 17, 2023

Related U.S. Application Data
(60) Provisional application No. 63/343,757, filed on May 19, 2022.

(51) Int. Cl. G06T 7/00; G06V 10/774; G06V 10/764

(52) U.S. Cl. CPC G06T 7/0014; G06V 10/774; G06V 10/764; G06T 2207/10081; G06T 2207/10096; G06T 2207/10132; G06T 2207/30096; G06T 2207/30048; G06T 2207/20081; G06T 2207/20084

(57) ABSTRACT

The invention provides systems and methods for evaluating and classifying one or more lesions formed in intravascular and/or intracardiac tissue for assisting in the diagnosis and/or treatment of a cardiac-related condition.

### CROSS-REFERENCE TO RELATED APPLICATIONS

This application claims priority to, and the benefit of, U.S. Provisional Application No. 63/343,757, filed on May 19, 2022, the content of which is incorporated by reference herein in its entirety.

### TECHNICAL FIELD

The disclosure relates to classification of biological tissue, and, more particularly, to systems and methods for evaluating and classifying one or more lesions formed in intravascular and/or intracardiac tissue for assisting in the diagnosis and/or treatment of a cardiac-related condition.

### BACKGROUND

Catheter ablation is a treatment in which energy is applied to cardiac tissue to create scars or lesions for preventing or interrupting the transmission of abnormal electrical signals. Catheter ablation forms an essential part of the management of cardiac arrhythmias, including supraventricular tachycardia (SVT), atrial flutter (AFL), atrial fibrillation (AF) and ventricular tachycardia (VT).

As the acceptance and performance of catheter ablation increases worldwide, limitations in current technologies are becoming increasingly apparent, particularly in the treatment of complex arrhythmias, such as AF. For example, with certain complex arrhythmia mechanisms, such as AF, arrhythmia recurrence is observed in over half of patients. The relatively low efficacy of AF treatment is likely due to limitations in mapping, incomplete understanding of the driving mechanisms of arrhythmia, and, most importantly, the inability to create transmural and durable lesions.

Successful catheter ablation requires not only precise localization of the arrhythmogenic substrate, but complete and permanent elimination of that substrate without producing collateral injury. The ablation effect depends on a number of factors, including applied electrical power, quality of the electrical contact, local tissue properties, presence of blood flow close to the tissue surface, and the effect of irrigation. Because of the variability of these parameters, it may be difficult to obtain consistent results and understand ablation effects in tissue using current systems and methods for ablation.

As a result, current ablation systems may be limited because of the difficulties and challenges in assessing the results of ablation in tissue, such as identifying a lesion formed in the tissue and determining various properties of the lesion through the catheter. Despite years of research and emergence of improved imaging technologies, the reliable creation of effective and permanent lesions remains challenging.

### SUMMARY

The present invention recognizes the drawbacks of current ablation systems, particularly the lack of reliable and accurate means of assessing lesion formation. As such, the present invention provides an image analysis platform configured to provide automated tissue evaluation and classification based on artificial intelligence techniques to address such drawbacks.

Aspects of the invention may be accomplished using a platform configured to analyze images acquired during a procedure, such as a catheter ablation procedure, and, in turn, identify, evaluate and classify one or more lesions formed in targeted tissue (i.e., intravascular and/or intracardiac tissue) in real, or near-real, time for assisting in the diagnosis and/or treatment of a cardiac-related condition.

In particular, the invention provides a computing system running a neural network that has been trained using a plurality of training data sets that include qualified reference data, which may include clinical data. For example, each training data set includes reference image data associated with known tissue and further includes classification data that is associated with the known tissue, wherein the plurality of training data sets excludes digital histopathology data. More specifically, the present invention recognizes that the assessment and validation of lesion formation is generally challenged by the need for histology. In particular, the common disadvantages and challenges of histology include time-consuming preparation (e.g., slicing, fixing, and staining of tissue on slides), inherent risk of human error during preparation and analysis of tissue samples, and lack of specificity with regard to conventional histological staining, which can affect its value as a diagnostic tool.

In an effort to overcome such challenges associated with histology, the present invention proposes the use of phase contrast computed tomography (CT) imaging as an alternative approach to using digital histopathology data as an input for training data. The present invention recognizes that the direct visualization of cardiac ablations via phase contrast CT may be particularly useful for bridging the gap between conventional imaging modality data (i.e., CT, magnetic resonance imaging (MRI), and ultrasound (US)) and histology, particularly with the assessment of lesion formation.

As such, in certain aspects of the present invention, the reference image data of the training data sets comprises one or more images of the known tissue obtained and processed via a phase contrast computed tomography (CT) imaging system and at least one other imaging modality, including, but not limited to, a transmission imaging system, a brightfield or darkfield imaging system, a fluorescence imaging system, a differential interference contrast imaging system, a hyperspectral imaging system, a Raman or surface-enhanced Raman imaging system, and a magnetic resonance imaging (MRI) system.

It should be noted that the reference image data relied upon for input for training data (i.e., phase contrast CT image data) has been validated, in that a link has been established between conventional histological data and the reference image data. In particular, the neural network is trained from the plurality of training data sets, such that the neural network is suitable for evaluating and classifying tissue based on an association of the classification data with the reference image data. Generally, the reference image data is associated with a reference lesion formed in a known tissue and the classification data is associated with the reference lesion. The classification data may include characteristics of the reference lesion, including, but not limited to, a location of the reference lesion on the known tissue, a size of the reference lesion, a pathway of the reference lesion, a depth of the reference lesion, and a known success of the reference lesion in the treatment of a cardiac-related condition.

In other words, each training data set is associated with a respective known tissue that has been collected as part of a clinical study or the like. The known tissue may have one or more known lesions therein. The known tissue (including known lesions formed therein) may undergo certain processes for the collection of both conventional histological data and the reference image data. In particular, reference image data may be collected from the clinical tissue sample (i.e., images of the clinical tissue sample may be acquired via phase contrast CT imaging system and/or other imaging modality described herein). Similarly, the clinical tissue sample may be prepared and analyzed via conventional histological techniques so as to collect conventional histological data. In turn, a link is established between the reference image data and the histological data, thereby validating what is shown in the reference image data (i.e., confirmation of what is shown in the image data based on the conventional histological techniques performed on the reference tissue samples, including the presence of any lesions and characteristics of such lesions).

The computing system is configured to receive tissue data of a patient, which may include image data acquired by an imaging modality used during a procedure. For example, during a catheter ablation procedure, the imaging modality may be an ultrasound imaging machine, in which ultrasound images of a target site are provided to the computing system. The target site may include a targeted area of intravascular and/or intracardiac tissue undergoing or having already undergone catheter ablation for the formation of one or more lesions to treat a cardiac condition, such as AF or the like.

It should be noted that the platform of the present invention may either be incorporated directly with an imaging modality (i.e., provided as a local component to an imaging machine or the like) or may be cloud-based and provide a digital, web-based application that an operator can access via an imaging system or computing device (i.e., smartphone, tablet, personal computer, or the like).

Upon receiving the image data of the patient undergoing a procedure, the computing system is configured to analyze the image data using the neural network and based on an association of classification data with the reference image data. Based on such analysis, the computing system is able to classify tissue within the images. In particular, the training data sets may include reference image data that is associated with a reference lesion formed in known tissue and the classification data is associated with the reference lesion. Accordingly, as part of the analysis, the computing system is able to correlate the image data of the patient with reference lesion and known tissue data, such that one or more lesion formations may be identified in the patient's image data and further classified. The classification of the identified one or more lesion formations may include various characteristics of the lesion formations that may be useful in assessing and validating the effectiveness of a lesion (i.e., predicting whether the lesion will successfully treat the condition). For example, the various characteristics identified may include, but are not limited to, a location of the one or more lesion formations on the sample tissue, a size of the one or more lesion formations, a pathway of the one or more lesion formations, a depth of the one or more lesion formations, and known success of the one or more lesion formations in the treatment of a cardiac-related condition.

Upon identifying and classifying tissue within the image data of the patient, including any lesion formations, the computing system further outputs the results of the tissue evaluation and classification to a user (i.e., a clinician or other medical professional associated with the patient). For example, in some embodiments, results may generally be provided to the user via a report, providing details about any lesion formations in the targeted tissue of the captured image.

Accordingly, by providing automated tissue evaluation and classification in real-time and based on artificial intelligence techniques, the present invention addresses the limitations of current ablation systems, particularly the lack of reliable and accurate means of assessing lesion formation. More specifically, the present invention reduces the need for any specialized training when evaluating image data to assess and validate lesion formation. Furthermore, the present invention does not require conventional histology in order to provide classification of lesions, which can present challenges, particularly with regard to complex registration processes and issues with deformation and/or tissue shifting that further impairs registration processes. Rather, the neural network of the present invention has been trained using a plurality of training data sets that include qualified reference data that excludes digital histopathology data. More specifically, the present invention utilized phase contrast CT image data as an alternative approach to using digital histopathology data as an input for training data. As such, the present invention provides a highly effective system for evaluating and classifying one or more lesions formed in intravascular and/or intracardiac tissue for assisting in the diagnosis and/or treatment of a cardiac-related condition.

One aspect of the present invention includes a method for training a neural network for evaluating and classifying tissue. The method includes providing, to a computing system, a plurality of training data sets, each training data set comprising reference image data associated with a known tissue and classification data associated with the known tissue, wherein the plurality of training data sets excludes digital histopathology data. The method further includes training a neural network from the plurality of training data sets such that the neural network is suitable for evaluating and classifying tissue based on an association of the classification data with the reference image data.

The reference image data may include one or more images of the known tissue obtained and processed via an imaging modality selected from the group consisting of: an ultrasound imaging system; a computed tomography (CT) imaging system; a transmission imaging system; a brightfield or darkfield imaging system; a fluorescence imaging system; a phase contrast imaging system; a differential interference contrast imaging system; a hyperspectral imaging system; a Raman or surface-enhanced Raman imaging system; and a magnetic resonance imaging (MRI) system. The MRI system may perform at least one of late gadolinium enhanced MRI and diffusion weighted MRI sequences.

In some embodiments, the reference image data may include images of the known tissue obtained and processed via an ultrasound imaging system and a CT imaging system. For example, the reference image data may include three-dimensional (3D) ultrasound image data and computed tomography (CT) image data of the known tissue. In some embodiments, the CT image data comprises phase contrast CT image data. For example, the CT image data may include post-mortem CT image data of the known tissue for anatomical reference and phase contrast CT image data of the known tissue. The 3D ultrasound image data may be obtained from a catheter-based ultrasound imaging device configured to provide full circumferential 3D image data.

In some embodiments, the reference image data may be associated with a reference lesion formed in the known tissue and the classification data is associated with the reference lesion. As such, the classification data may include characteristics of the reference lesion including at least one of: a location of the reference lesion on the known tissue; a size of the reference lesion; a pathway of the reference lesion; a depth of the reference lesion, and a known success of the reference lesion in the treatment of a cardiac-related condition.

The method may further include the steps of obtaining one or more images of sample tissue undergoing an ablation procedure, processing the one or more images and inputting sample image data, obtained in the processing step, into the computing system, correlating the sample image data with the reference lesion and known tissue data, and outputting results of the correlating step. The results of the correlating step may include identification of one or more lesion formations in the sample tissue and classification of the identified one or more lesion formations. As such, classification of the identified one or more lesion formations may include identified characteristics including at least one of: a location of the one or more lesion formations on the sample tissue; a size of the one or more lesion formations; a pathway of the one or more lesion formations; a depth of the one or more lesion formations; and known success of the one or more lesion formations in the treatment of a cardiac-related condition. It should be noted that, in some embodiments, the method may further include providing a binary classification of tissue (i.e., ablated and non-ablated portions). The method may further include providing lesion mapping, which may generally include providing a likelihood or probability of lesion formations as a 3D image representation (i.e., reconstructing a full 3D volume, such that each point in the 3D volume includes a probability of tissue being ablated or not ablated). The results of the correlating step further may further include validation of one or more lesion formations. In some embodiments, the computing system may include a machine learning system selected from the group consisting of a neural network, a random forest, a support vector machine, a Bayesian classifier, a Hidden Markov model, an independent component analysis method, and a clustering method. For example, in some embodiments, the computing system may include an autonomous machine learning system that associates the classification data with the reference image data. In some embodiments, the machine learning system may include a deep learning neural network that includes an input layer, a plurality of hidden layers, and an output layer. Yet still, in some embodiments, the autonomous machine learning system may represent the training data set using a plurality of features, wherein each feature comprises a feature vector. In some embodiments, the autonomous machine learning system may include a convolutional neural network.

In some embodiments, the method may further include the step of operating a machine learning system to learn relationships among reference image data, classification data, and lesion formation via ablation procedures.

### BRIEF DESCRIPTION OF THE DRAWINGS

FIGS. 1A and 1B are diagrammatic illustrations of an exemplary medical imaging machine that is compatible for use with systems and methods of the present invention, including a system for providing automated tissue evaluation and classification.

FIG. 2 is a block diagram illustrating a system for providing automated tissue evaluation and classification, wherein the system uses machine learning techniques for classifying lesions formed in intravascular and/or intracardiac tissue based on image analysis.

FIG. 3 is a block diagram illustrating an automated tissue evaluation and classification system, including a machine learning system, consistent with the present disclosure.

FIG. 4 is a block diagram illustrating inputting of reference data (i.e., training data sets) into the machine learning system.

FIG. 5 shows a machine learning system according to certain embodiments of the present disclosure.

FIG. 6 is a flow diagram illustrating an exemplary process for collecting, processing, and inputting reference data into the machine learning system of the present invention.

FIG. 7 is a block diagram illustrating receipt of one or more images acquired during a procedure (such as an ablation procedure) performed on targeted tissue of a patient, subsequent processing of the images via an automated tissue evaluation and classification system of the present invention, and outputting of results to an operator (i.e., clinician performing or assisting with the ablation procedure), wherein such results include, among other things, identification of one or more lesion formations in the targeted tissue, and classification of such lesion formation to assist in the diagnosis and/or treatment of an associated condition.

FIG. 8 shows an image of a sample tissue having undergone histological analysis and a corresponding reference image (i.e., a phase contrast image) of the sample tissue, in which a link has been established for use in validating the reference image.

### DETAILED DESCRIPTION

By way of overview, the present invention is directed to systems and methods for classifying biological tissue, including automated tissue evaluation and classification based on artificial intelligence techniques. More specifically, aspects of the invention may be accomplished using a platform configured to analyze images of a target site of a patient undergoing a procedure and, in turn, identify features of tissue in the images, including identification and classification of lesion formations at the target site.

In particular, the platform is configured to receive input in the form of image data acquired by an imaging modality used during a procedure. For example, during a catheter ablation procedure, the imaging modality may be an ultrasound imaging machine, in which ultrasound images of a target site are provided to the platform. The target site may include a targeted area of intravascular and/or intracardiac tissue undergoing or having already undergone catheter ablation for the formation of one or more lesions to treat a cardiac condition, such as AF or the like.

The platform is configured to analyze the image data using the neural network, relying on pre-trained data sets containing reference image data associated with known tissue and classification data that is associated with the known tissue, wherein the plurality of training data sets excludes digital histopathology data. More specifically, the reference image data of the training data sets comprises one or more images of the known tissue obtained and processed via a phase contrast computed tomography (CT) imaging system and at least one other imaging modality, including, but not limited to, an ultrasound imaging system, a transmission imaging system, a brightfield or darkfield imaging system, a fluorescence imaging system, a differential interference contrast imaging system, a hyperspectral imaging system, a Raman or surface-enhanced Raman imaging system, and a magnetic resonance imaging (MRI) system.

In the current example of a catheter ablation procedure, in which the goal is successfully create lesions resulting in complete and permanent elimination of a target tissue, or portion thereof, the training data sets may include reference image data that is associated with a reference lesion formed in known tissue and the classification data is associated with the reference lesion. Accordingly, as part of the analysis, the platform is able to correlate the image data of the patient with reference lesion and known tissue data, such that one or more lesion formations may be identified in the patient's image data and further classified. The classification of the identified one or more lesion formations may include various characteristics of the lesion formations that may be useful in assessing and validating the effectiveness of a lesion (i.e., predicting whether the lesion will successfully treat the condition). For example, the various characteristics identified may include, but are not limited to, a location of the one or more lesion formations on the sample tissue, a size of the one or more lesion formations, a pathway of the one or more lesion formations, a depth of the one or more lesion formations, and known success of the one or more lesion formations in the treatment of a cardiac-related condition.

Upon identifying and classifying tissue within the image data of the patient, including any lesion formations, the platform further outputs the results of the tissue evaluation and classification to a user (i.e., a clinician or other medical professional associated with the patient). For example, in some embodiments, results may generally be provided to the user via a report, providing details about any lesion formations in the targeted tissue of the captured image.

Accordingly, by providing automated tissue evaluation and classification in real-time and based on artificial intelligence techniques, the present invention addresses the limitations of current ablation systems, particularly the lack of reliable and accurate means of assessing lesion formation. More specifically, the present invention reduces the need for any specialized training when evaluating image data to assess and validate lesion formation. Furthermore, the present invention does not require conventional histology in order to provide classification of lesions, which can present challenges, particularly with regard to complex registration processes and issues with deformation and/or tissue shifting that further impairs registration processes. Rather, the neural network of the present invention has been trained using a plurality of training data sets that include qualified reference data that excludes digital histopathology data. More specifically, the present invention utilized phase contrast CT image data as an alternative approach to using digital histopathology data as an input for training data. As such, the present invention provides a highly effective system for evaluating and classifying one or more lesions formed in intravascular and/or intracardiac tissue for assisting in the diagnosis and/or treatment of a cardiac-related condition.

It should be noted that, while the following description focuses on use of the present invention for catheter ablation procedures and classifying lesion formations associated therewith, the systems and methods of the present invention can be used for classifying tissue of any kind with respect to any kind of procedure in which imaging analysis is used and/or preferred.

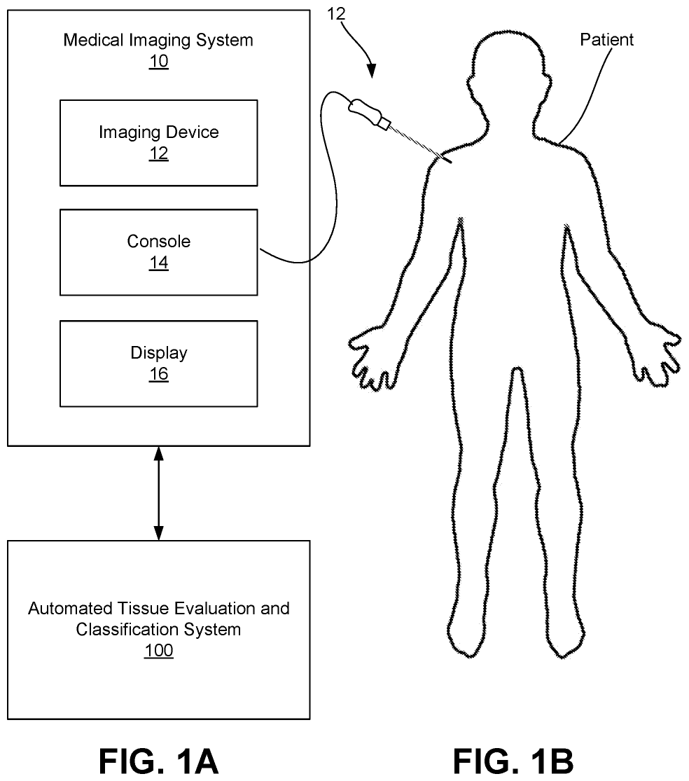

FIGS. 1A and 1B are diagrammatic illustrations of an exemplary medical imaging system that is compatible for use with systems and methods of the present invention, including a system 100 for providing automated tissue evaluation and classification. The medical imaging system 10 may include any type of medical imaging modality, including, but not limited to, an ultrasound imaging system, a computed tomography (CT) imaging system, a transmission imaging system, a brightfield or darkfield imaging system, a fluorescence imaging system, a phase contrast imaging system, a differential interference contrast imaging system, a hyperspectral imaging system, a Raman or surface-enhanced Raman imaging system, and a magnetic resonance imaging (MRI) system.

In the illustrated embodiment, the system 10 is an ultrasound system and includes an imaging device 12 operably coupled to a console 14 and a display 16. As generally understood, ultrasound imaging (sonography) uses high-frequency sound waves to view inside the body. Because ultrasound images are captured in real-time, they can also show movement of the body's internal organs as well as fluid flow (e.g., blood flowing through blood vessels). In an ultrasound exam, the ultrasound device 12, also referred to as a transducer probe, is placed directly on the skin or inside a body opening.

The ultrasound transducer probe 12 is responsible for sending and receiving the sound waves that create an ultrasound image via the piezoelectric effect, a phenomenon that causes quartz crystals within the probe to vibrate rapidly and send out sound waves. These waves then bounce off objects and are reflected to the probe.

The transducer probe 12 is operably coupled to a console 14, which generally controls operation of the transducer probe 12 (i.e., transmission of sound waves from the probe). The console 14 may generally include a central processing unit (CPU), storage, and some form of input (i.e., a keyboard, knobs, scroll wheels, or the like) with which an operator can interact so as to operate the machine, including making adjustments to the transmission characteristics of the probe, saving images, and performing other tasks. During operation, the CPU transmits electrical currents that cause the probe 12 to emit sound waves. The CPU also analyzes electrical pulses that the probe makes in response to reflected waves coming back. It then converts this data into images (i.e., ultrasound images) that can then be viewed on a display 16, which may be an integrated monitor. Such images may also be stored in memory and/or printed via a printer (not shown).

In the illustrated embodiment, the imaging device 12 may generally be in the form of an imaging catheter that may be capable of providing imaging and mapping capabilities, and, in some embodiments, energy delivery (i.e., ablation). Accordingly, such a device 12 may be useful in carrying out catheter ablation to treat a cardiac condition, such as AF or the like. For example, in some embodiments, the catheter 12 may further include additional components providing associated capabilities. For example, portions of the catheter may include sensors (e.g., localization and/or tracking sensors) and/or energy delivery elements (e.g., ablation elements).

The imaging catheter 12 may include a fully rotatable transducer unit comprised of an ultrasound transducer array configured to transmit ultrasound pulses to, and receive echoes of the ultrasound pulses from, surrounding intravascular tissue during a procedure. Such ultrasound transmissions result in a collection of image data which is received by the console 14 and subsequently reconstructed into one or more images providing visualization and characterization of the surrounding intravascular tissue. In particular, the console 14 may utilize image data received from an imaging assembly of the imaging catheter 12 to reconstruct one or more images providing full circumferential, 360-degree visualization of the intravascular tissue. The console 14 may process the received image data utilizing certain imaging protocols and algorithms for reconstructing images and subsequently outputting, via a display, the reconstructed images (two-, three-, or four-dimensional images) to an operator depicting visualization of the intravascular tissue, In addition to providing reconstruction of images based on received image data from the imaging assembly, the console 14 may further provide control over the imaging assembly, including control over the emission of ultrasound pulses therefrom (intensity, frequency, duration, etc.) as well as control over the movement of the ultrasound transducer unit (i.e., controlling rotation, including speed and duration of rotation). For example, the console 14 may be equipped with certain hardware and software for providing such image reconstruction and imaging assembly control, as described in International PCT Application No. PCT/M2019/000963 (Published as WO 2020/044117) to Hennersperger et al., the content of which is incorporated by reference herein in its entirety.

As previously described, the present invention is directed to systems and methods for classifying biological tissue, including automated tissue evaluation and classification based on artificial intelligence techniques. More specifically, aspects of the invention may be accomplished using a platform configured to analyze images of a target site of a patient undergoing a procedure and, in turn, identify features of tissue in the images, including identification and classification of lesion formations at the target site.

As shown in FIG. 1A, the invention may include an automated tissue evaluation and classification system 100. This system 100 may either be incorporated directly with an ultrasound machine (i.e., provided as a local component to an ultrasound imaging machine) or may be cloud-based and provide a digital, web-based application that an operator can access via an ultrasound machine or computing device (i.e., smartphone, tablet, personal computer, or the like).

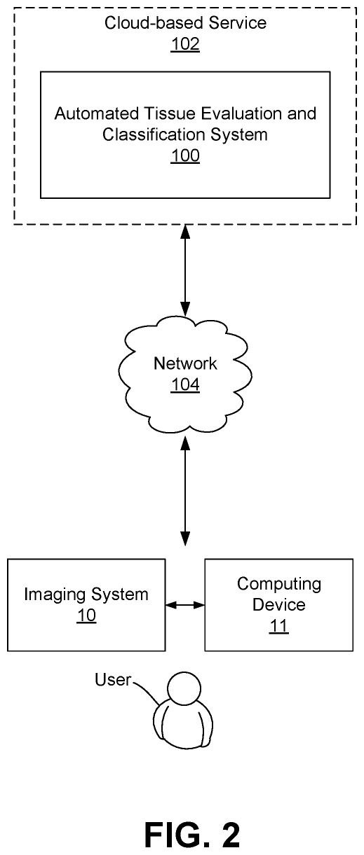

FIG. 2 is a block diagram illustrating automated tissue evaluation and classification system 100 in greater detail. As shown, the system 100 is embodied on a cloud-based service 102, for example. The automated ultrasound imaging analysis system 100 is configured to communicate and share data with an ultrasound imaging machine 10. It should be noted, however, that the system 100 may also be configured to communicate and share data with a computing device 11 associated with a user. The computing device 11 may include a separate computer coupled to an ultrasound imaging machine. Yet still, in some embodiments, the computing device 11 may include a portable computing device, such as a smartphone, tablet, laptop computer, or the like. For example, advances in technology have led to some ultrasound probes being connectable to a personal and/or portable computing device. Accordingly, in some embodiments, the system 100 may be configured to communicate with an operator of an ultrasound probe via an associated smartphone or tablet. In the present context, the user may include a clinician, such as a physician, physician's assistant, nurse, or other medical professional providing an ultrasound examination on a patient and/or catheter ablation procedure. The system 100 is configured to communicate and exchange data with the ultrasound imaging machine 10 and/or computing device 11 over a network 104, for example.

The network 104 may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the ultrasound imaging machine 10 and computing device 11 and/or between the machine 10, computing device 11 and system 100 may be, in whole or in part, a wired connection.

The network 104 may be any network that carries data. Non-limiting examples of suitable networks that may be used as network 18 include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G), fifth generation (5G), and future generations of cellular-based data communication technologies, Bluetooth radio, Near Field Communication (NFC), the most recently published versions of IEEE 802.11 transmission protocol standards, other networks capable of carrying data, and combinations thereof. In some embodiments, network 104 is chosen from the internet, at least one wireless network, at least one cellular telephone network, and combinations thereof. As such, the network 104 may include any number of additional devices, such as additional computers, routers, and switches, to facilitate communications. In some embodiments, the network 104 may be or include a single network, and in other embodiments the network 104 may be or include a collection of networks.

It should be noted that, in some embodiments, the system 100 is embedded directly into an ultrasound machine, or may be directly connected thereto in a local configuration, as opposed to providing a web-based application. For example, in some embodiments, the system 100 operates in communication with a medical setting, such as an examination or procedure room, laboratory, or the like, may be configured to communicate directly with instruments, including, for example, the ultrasound imaging machine 10 either via a wired or wireless connection.

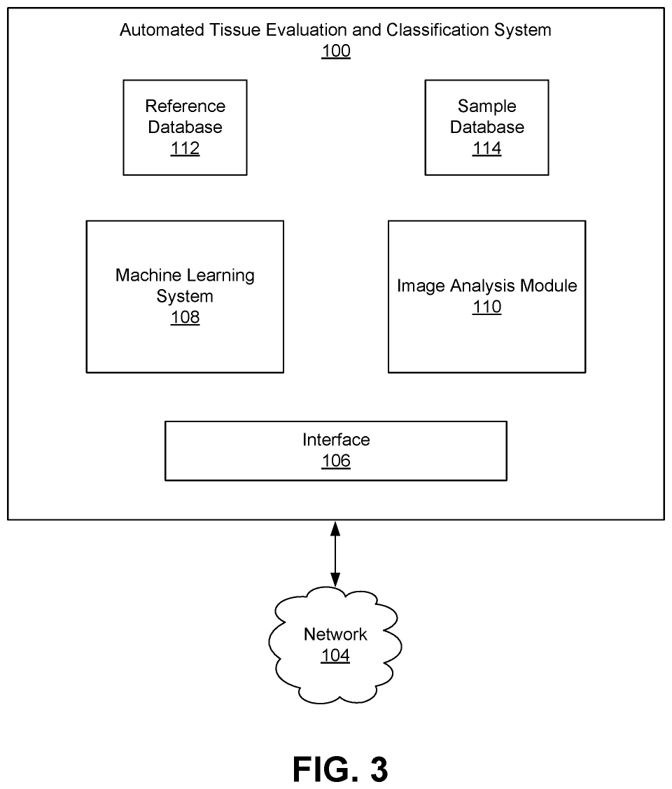

FIG. 3 is a block diagram illustrating the automated tissue evaluation and classification system 100, including a machine learning system 108, for providing the automated analysis of image data and providing subsequent identification, evaluation, and classification of tissue in the image data. The system 100 is preferably implemented in a tangible computer system built for implementing the various methods described herein.

As illustrated, the system 100 may generally be accessed by a user, to initiate methods of the invention and obtain results, via an interface 106, for example. The interface 106 allows for a user to connect with the platform provided via the system and provide sample ultrasound images to undergo automated analysis and feedback. The system 100 may further include one or more databases with which the machine learning system 108 communicates. In the present example, a reference database 112 includes stored reference data obtained from a plurality of training data sets and a sample database 114 includes stored sample data acquired as a result of evaluations carried out via the system 100 on sample ultrasound images. The system 100 further includes an image analysis module 110 to assist in the processing of input image data and correlating such data with reference image data of trained data sets of the neural network.

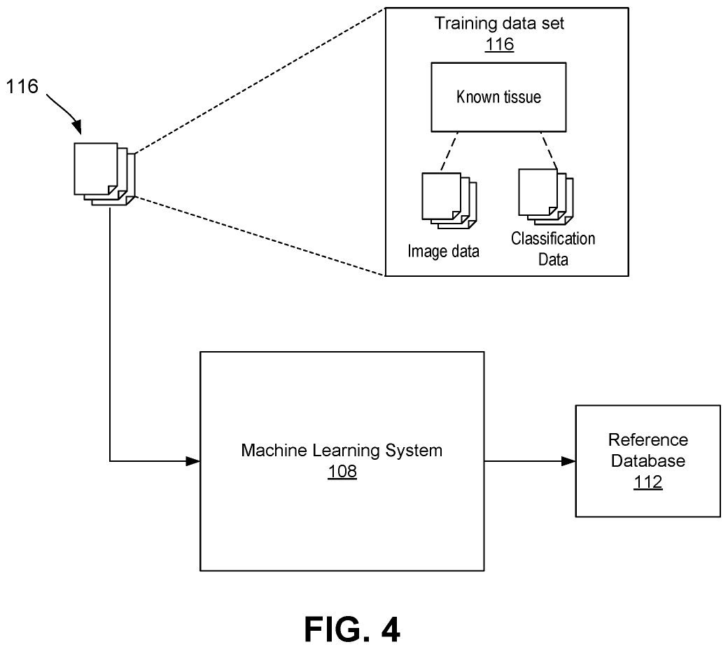

The system 100 generally runs a neural network that has been trained using a plurality of training data sets that include qualified reference data. FIG. 4 is a block diagram illustrating inputting of reference data (i.e., training data sets) into the machine learning system 108, for example. The machine learning techniques of the present invention, and the subsequent analysis of sample ultrasound images based on such techniques, utilize reference data. The reference data may include a plurality of training data sets 116 inputted to a machine learning system 108 of the present invention. For example, each training data set includes reference image data associated with known tissue and further includes classification data that is associated with the known tissue, wherein the plurality of training data sets excludes digital histopathology data. More specifically, the present invention recognizes that the assessment and validation of lesion formation is generally challenged by the need for histology. In effort to overcome such challenges, the present invention proposes the use of phase contrast computed tomography (CT) imaging as an alternative approach to using digital histopathology data as an input for training data. The present invention recognizes that the direct visualization of cardiac ablations via phase contrast CT may be particularly useful for bridging the gap between conventional imaging modality data (i.e., CT, magnetic resonance imaging (MRI), and ultrasound (US)) and histology, particularly with the assessment of lesion formation.

As such, in certain aspects of the present invention, the reference image data of the training data sets comprises one or more images of the known tissue obtained and processed via a phase contrast computed tomography (CT) imaging system and at least one other imaging modality, including, but not limited to, an ultrasound imaging system, a transmission imaging system, a brightfield or darkfield imaging system, a fluorescence imaging system, a differential interference contrast imaging system, a hyperspectral imaging system, a Raman or surface-enhanced Raman imaging system, and a magnetic resonance imaging (MRI) system.

In the present example, the reference image data is associated with a reference lesion formed in the known tissue and the classification data is associated with the reference lesion. Accordingly, the classification data may include characteristics of the reference lesion including at least one of: a location of the reference lesion on the known tissue; a size of the reference lesion; a pathway of the reference lesion; a depth of the reference lesion, and a known success of the reference lesion in the treatment of a cardiac-related condition.

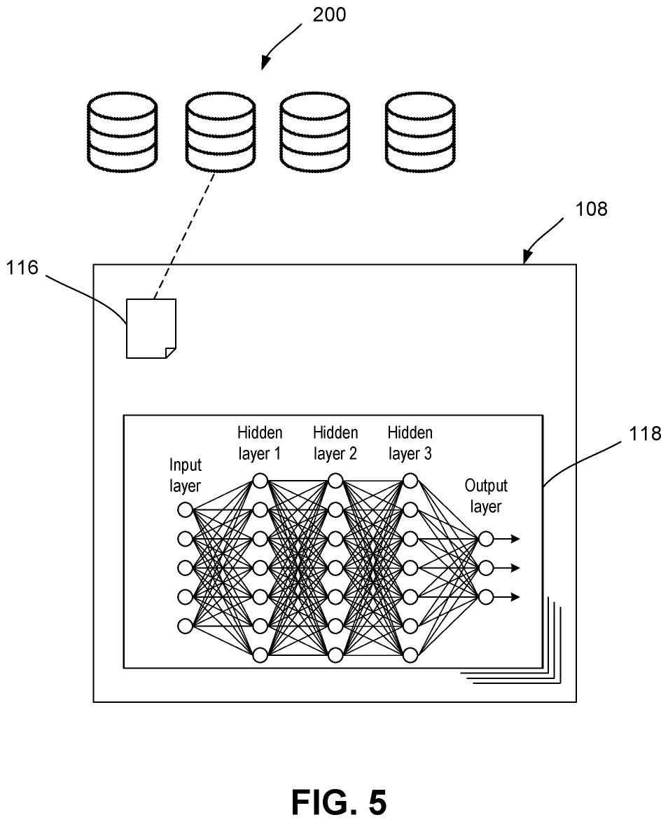

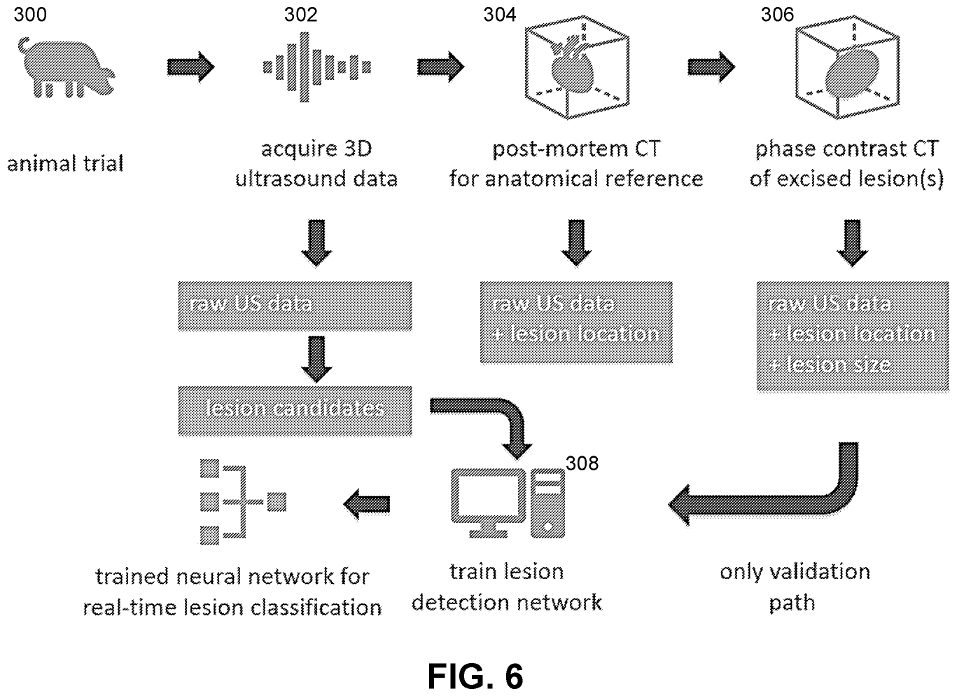

FIG. 5 shows a machine learning system according to certain embodiments of the present disclosure. The machine learning system 108 accesses reference data from the one or more training data sets 116 provided by any known source 200. The source 200 may include, for example, a laboratory-specific repository of reference data collected for purposes of machine learning training. Additionally, or alternatively, the source 200 may include publicly available registries and databases and/or subscription-based data sources. In the present example, the source 200 is clinical data, as further described in greater detail herein with reference to FIG. 6.

In preferred embodiments, the plurality of training data sets 116 feed into the machine learning system 108. The machine learning system 108 may include, but is not limited to, a neural network, a random forest, a support vector machine, a Bayesian classifier, a Hidden Markov model, an independent component analysis method, and a clustering method.

For example, the machine learning system 108 an autonomous machine learning system that associates the classification data with the reference image data. For example, the machine learning system may include a deep learning neural network that includes an input layer, a plurality of hidden layers, and an output layer. The autonomous machine learning system may represent the training data set using a plurality of features, wherein each feature comprises a feature vector. For example, the autonomous machine learning system may include a convolutional neural network (CNN). In the depicted embodiment, the machine learning system 108 includes a neural network 118.

The machine learning system 108 discovers associations in data from the training data sets. In particular, the machine learning system 108 processes and associates the reference image data and classification data with one another, thereby establishing reference data in which image characteristics of known tissue, including lesions, are associated with known characteristics of the tissue and lesions, a location of the reference lesion on the known tissue, a size of the reference lesion, a pathway of the reference lesion, a depth of the reference lesion, and a known success of the reference lesion in the treatment of a cardiac-related condition. In particular, the machine learning system 108 is able to learn relationships among reference image data, classification data, and lesion formation via ablation procedures. The reference data is stored within the reference database 112, for example, and available during subsequent processing of acquired image data during a catheter ablation procedure.

FIG. 6 is a flow diagram illustrating an exemplary process for collecting, processing, and inputting reference data into the machine learning system of the present invention. The reference data is generally clinical data that has been collected via animal trials 300.

Such reference data includes acquiring in-vivo image data 302, which may include, for example, one or more images of a known tissue having one or more known lesions formed therein. The image data may be acquired by any contemplated imaging modality described herein, including, but not limited to, an ultrasound imaging system, a computed tomography (CT) imaging system, a transmission imaging system, a brightfield or darkfield imaging system, a fluorescence imaging system, a phase contrast imaging system, a differential interference contrast imaging system, a hyperspectral imaging system, a Raman or surface-enhanced Raman imaging system, and a magnetic resonance imaging (MRI) system. In a preferred embodiment, the reference image data acquired in-vivo is ultrasound image data, and, in some embodiments, three-dimensional (3D) ultrasound image data.

In some embodiments, additional reference data may include acquiring post-mortem image data 304 for use as an anatomical reference, wherein such image data may include computed tomography (CT) image data of the known tissue (i.e., the known tissue having one or more known lesions formed therein). For example, in some embodiments, it may be preferable to acquire post-mortem video data for the annotation of known lesions (e.g., with TTC staining) wherein such post-mortem acquired data can be associated (via mapping techniques) with the in-vivo acquired image data to the specific lesion or set of lesions at a given target site (i.e., lesion location or the like).

Yet still, in some embodiments, additional reference data may include acquiring post-mortem-image data, including phase contrast CT image data, of specific excised lesions 306 that are mapped to the in-vivo image data.

With each set of reference data (302, 304, and 306) certain characteristics are known, which may include, a location of the reference lesion on the known tissue, a size of the reference lesion, a pathway of the reference lesion, a depth of the reference lesion, and a known success of the reference lesion in the treatment of a cardiac-related condition (i.e., whether a given reference lesion is transmural, complete, and/or durable so as to effectively treat the underlying condition).

The machine learning system 108 discovers associations in data from the training data sets 308. In particular, the machine learning system 108 processes and associates the reference image data and classification data with one another, thereby establishing reference data in which image characteristics of known tissue, including lesions, are associated with classification data.

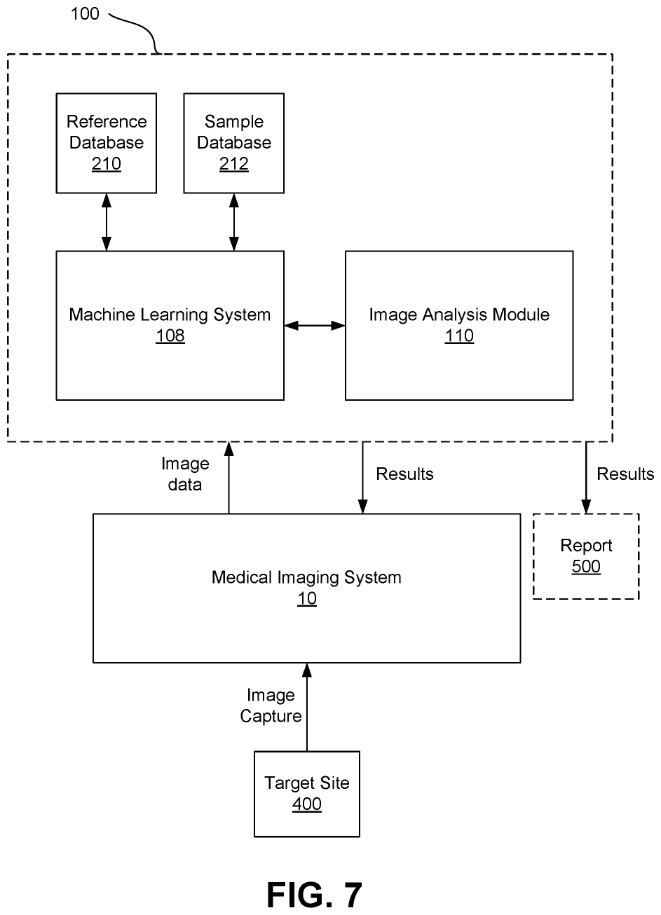

FIG. 7 is a block diagram illustrating receipt of one or more images acquired during a procedure (such as an ablation procedure) performed on targeted tissue of a patient, subsequent processing of the images via an automated tissue evaluation and classification system 100 of the present invention, and outputting of results to an operator (i.e., clinician performing or assisting with the ablation procedure), wherein such results include, among other things, identification of one or more lesion formations in the targeted tissue, and classification of such lesion formations to assist in the diagnosis and/or treatment of an associated condition.

As shown, the system 100 is configured to receive image data, which may generally include images (obtained via a medical imaging modality 10) of a target site of a patient undergoing a procedure. In the present example, the images are ultrasound images captured via an ultrasound image system with an ablation catheter, for example. Upon receiving the ultrasound images, the system 100 is configured to analyze the images using the neural network of the machine learning system 108 and based on an association of classification data with the reference image data. Based on such analysis, the computing system is able to evaluate and classify one or more lesions that are formed in tissue at the target site undergoing catheter ablation. More specifically, the machine learning system 108 correlates the sample ultrasound image data with the reference data (i.e., the reference image data and classification data). For example, the machine learning system 108 may include custom, proprietary, known and/or after-developed statistical analysis code (or instruction sets), hardware, and/or firmware that are generally well-defined and operable to receive two or more sets of data and identify, at least to a certain extent, a level of correlation and thereby associate the sets of data with one another based on the level of correlation.

Upon detecting that association among ultrasound image data and the reference image data and classification data, the system 100 is able to output the results to a clinician. The results may be in the form of a report 500, for example, which provides details about the target site, namely and identification of lesion formations and specific characteristics of each. For example, the results may be provided to the clinician via the display 16 of the imaging modality 10, which may include an annotated image of the target site, including a visual rendering of the lesion formations and characteristics of each.

Accordingly, by providing automated tissue evaluation and classification in real-time and based on artificial intelligence techniques, the present invention addresses the limitations of current ablation systems, particularly the lack of reliable and accurate means of assessing lesion formation. More specifically, the present invention reduces the need for any specialized training when evaluating image data to assess and validate lesion formation. Furthermore, the present invention does not require conventional histology in order to provide classification of lesions, which can present challenges, particularly with regard to complex registration processes and issues with deformation and/or tissue shifting that further impairs registration processes. Rather, the neural network of the present invention has been trained using a plurality of training data sets that include qualified reference data that excludes digital histopathology data. More specifically, the present invention utilized phase contrast CT image data as an alternative approach to using digital histopathology data as an input for training data. As such, the present invention provides a highly effective system for evaluating and classifying one or more lesions formed in intravascular and/or intracardiac tissue for assisting in the diagnosis and/or treatment of a cardiac-related condition.

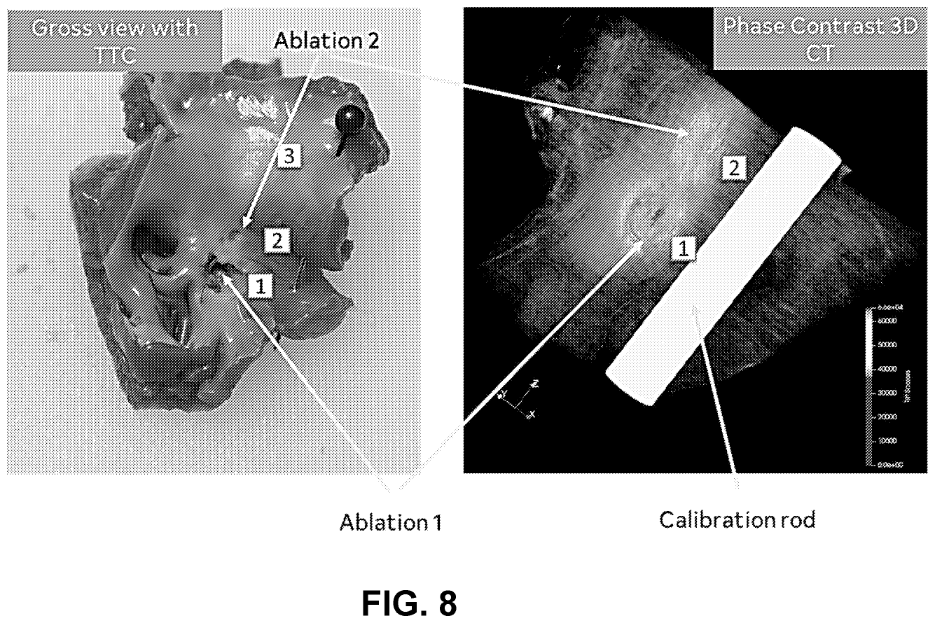

FIG. 8 shows an image of a sample tissue having undergone histological analysis and a corresponding reference image (i.e., a phase contrast image) of the sample tissue. As previously described herein, the reference image data relied upon for input for training data (i.e., phase contrast CT image data) is validated, in that a link is established between conventional histological data and the reference image data. In particular, each training data set is associated with a respective known tissue that has been collected as part of a clinical study or the like. The known tissue may have one or more known lesions therein. The known tissue (including known lesions formed therein) may undergo certain processes for the collection of both conventional histological data and the reference image data. In particular, reference image data may be collected from the clinical tissue sample (i.e., images of the clinical tissue sample may be acquired via phase contrast CT imaging system and/or other imaging modality described herein). Similarly, the clinical tissue sample may be prepared and analyzed via conventional histological techniques so as to collect conventional histological data. In turn, a link is established between the reference image data and the histological data, thereby validating what is shown in the reference image data (i.e., confirmation of what is shown in the image data based on the conventional histological techniques performed on the reference tissue samples, including the presence of any lesions and characteristics of such lesions).

As shown in FIG. 8, the reference image (phase contrast CT image) can be validated based on the histological analysis performed on the sample tissue, in which ablations (ablation 1 and ablation 2) identified as part of the histological analysis are linked to the ablations shown in the phase contrast CT image. Accordingly, a link is established between histology/staining and the phase contrast CT image, thereby confirming the accuracy of the visual representation of the reference image (i.e., confirmation of what is shown in the image data based on the conventional histological techniques performed on the reference tissue samples, including the presence of any lesions and characteristics of such lesions). The link can be established by using a specific calibration feature, such as a calibration rod, which allows for a mapping of tissue properties to measured phase contrast CT data in an absolute fashion. By using a calibration rod, absolute tissue properties can be directly mapped, and thereby improve visualizations of ablations.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for training a neural network for evaluating and classifying tissue, the method comprising:
providing, to a computing system, a plurality of training data sets, each training data set comprising reference image data associated with a known tissue and classification data associated with the known tissue, wherein the plurality of training data sets excludes digital histopathology data; and
training a neural network from the plurality of training data sets such that the neural network is suitable for evaluating and classifying tissue based on an association of the classification data with the reference image data.

2. The method of claim 1, wherein the reference image data comprises one or more images of the known tissue obtained and processed via an imaging modality selected from the group consisting of: an ultrasound imaging system; a computed tomography (CT) imaging system; a transmission imaging system; a brightfield or darkfield imaging system; a fluorescence imaging system; a phase contrast imaging system; a differential interference contrast imaging system; a hyperspectral imaging system; a Raman or surface-enhanced Raman imaging system, and a magnetic resonance imaging (MRI) system.

3. The method of claim 2, wherein the MRI system performs at least one of late gadolinium enhanced MRI and diffusion weighted MRI sequences.

4. The method of claim 2, wherein the reference image data comprises images of the known tissue obtained and processed via an ultrasound imaging system and a CT imaging system.

5. The method of claim 4, wherein the reference image data comprises three-dimensional (3D) ultrasound image data and computed tomography (CT) image data of the known tissue, wherein the CT image data comprises post-mortem CT image data of the known tissue for anatomical reference and phase contrast CT image data of the known tissue.

6. The method of claim 1, wherein the reference image data is associated with a reference lesion formed in the known tissue and the classification data is associated with the reference lesion, wherein the classification data comprises characteristics of the reference lesion including at least one of: a location of the reference lesion on the known tissue; a size of the reference lesion; a pathway of the reference lesion; a depth of the reference lesion, and a known success of the reference lesion in the treatment of a cardiac-related condition.

7. The method of claim 6, further comprising:
obtaining one or more images of sample tissue undergoing an ablation procedure;
processing the one or more images and inputting sample image data, obtained in the processing step, into the computing system;
correlating the sample image data with the reference lesion and known tissue data; and
outputting results of the correlating step, wherein the results of the correlating step comprises identification of one or more lesion formations in the sample tissue and classification of the identified one or more lesion formations based on identified characteristics of the one or more lesion formations.

8. The method of claim 7, wherein the results of the correlating step further comprise validation of one or more lesion formations.

9. The method of claim 1, wherein the computing system comprises an autonomous machine learning system that associates the classification data with the reference image data, wherein the machine learning system comprises a deep learning neural network that includes an input layer, a plurality of hidden layers, and an output layer.

10. The method of claim 9, wherein the autonomous machine learning system represents the training data set using a plurality of features, wherein each feature comprises a feature vector.

11. A method for classifying tissue, the method comprising:
providing tissue data of a patient to a computer running a neural network, wherein the neural network has been trained to classify tissue and the neural network has been trained using a plurality of training data sets, each training data set comprising reference image data associated with a known tissue and known classification data associated with the known tissue, wherein the plurality of training data sets excludes digital histopathology data; and
classifying the tissue data of the patient using the neural network and based on an association of the classification data with the reference image data.

12. The method of claim 11, wherein the reference image data comprises one or more images of the known tissue obtained and processed via an imaging modality selected from the group consisting of: an ultrasound imaging system; a computed tomography (CT) imaging system; a transmission imaging system; a brightfield or darkfield imaging system; a fluorescence imaging system; a phase contrast imaging system; a differential interference contrast imaging system; a hyperspectral imaging system; a Raman or surface-enhanced Raman imaging system, and a magnetic resonance imaging (MRI) system.

13. The method of claim 12, wherein the MRI system performs at least one of late gadolinium enhanced MRI and diffusion weighted Mill sequences.

14. The method of claim 12, wherein the reference image data comprises images of the known tissue obtained and processed via an ultrasound imaging system and a CT imaging system.

15. The method of claim 14, wherein the reference image data comprises three-dimensional (3D) ultrasound image data and computed tomography (CT) image data of the known tissue, wherein the CT image data comprises post-mortem CT image data of the known tissue for anatomical reference and phase contrast CT image data of the known tissue.

16. The method of claim 11, wherein the reference image data is associated with a reference lesion formed in the known tissue and the classification data is associated with the reference lesion, wherein the classification data comprises characteristics of the reference lesion including at least one of: a location of the reference lesion on the known tissue; a size of the reference lesion; a pathway of the reference lesion; a depth of the reference lesion, and a known success of the reference lesion in the treatment of a cardiac-related condition.

17. The method of claim 16, further comprising:
obtaining one or more images of sample tissue undergoing an ablation procedure;
processing the one or more images and inputting sample image data, obtained in the processing step, into the computing system;
correlating the sample image data with the reference lesion and known tissue data; and
outputting results of the correlating step, wherein the results of the correlating step comprise identification of one or more lesion formations in the sample tissue and classification of the identified one or more lesion formations based on identified characteristics of the one or more lesion formations.

18. The method of claim 17, wherein the results of the correlating step further comprise validation of one or more lesion formations.

19. The method of claim 11, wherein the computing system comprises an autonomous machine learning system that associates the classification data with the reference image data, wherein the machine learning system comprises a deep learning neural network that includes an input layer, a plurality of hidden layers, and an output layer.

20. The method of claim 19, wherein the autonomous machine learning system represents the training data set using a plurality of features, wherein each feature comprises a feature vector.
